(19) United States
(12) Patent Application Publication
(10) Pub. No.: US 2008/0085270 A1
(43) Pub. Date: Apr. 10, 2008

(54) CoEnzyme Q10 formulation

(76) Inventor: Wayne F. Gorsek, Boynton Beach, FL (US)

Correspondence Address: Christopher J. McDonald;HOFFMAN, WASSON & GITLER, P.C., Suite 522, 2461 South Clark Street, Arlington, VA, 22202, US

(21) Appl. No.: 11/544,568

(22) Filed: Oct. 10, 2006

(51) Int. Cl. A61K 38/43; A61K 36/67; A61K 36/899; A61K 31/355

(52) U.S. Cl. 424/94.1; 424/750; 424/734; 514/458

(57) ABSTRACT

The formula contains a combination of nutrients ideal for cardiovascular health. Coenzyme Q10 (CoQ10), tocotrienols and tocopherols (potent forms of vitamin E) promote healthy heart function, provide powerful antioxidant protection and support the immune system in its daily defense of the body. The formula also contains a unique tocotrienol complex, derived from the fruit oil of palm trees. Tocotrienols (also known as “super vitamin E”) have been clinically proven to promote healthy cholesterol levels. They inhibit production of cholesterol in the liver and may actually be able to reverse arterial blockage, promoting efficient blood flow supply to the heart muscle.

### BACKGROUND OF THE INVENTION

Coenzyme Q10 (CoQ10), or ubiquinone, is a naturally-occurring nutrient found in cell mitochondria, where energy is produced. The heart, with greater energy requirements than other organs, contains significantly higher amounts of CoQ10 than other tissues in the body. Unfortunately, levels of CoQ10 naturally decline with age, which may result in less efficient functioning of the heart and other organs.

Besides supporting cardiovascular health, CoQ10 has been shown to benefit a number of other health concerns. A study published in the Archives of Neurology found that supplementation with up to 1,200 mg of CoQ10 daily may slow the progression of Parkinson's disease. Other studies suggest that CoQ10 may be associated with periodontal (gum), immune and circulatory health, as individuals with concerns in these areas were found to be deficient in CoQ10.

### SUMMARY OF THE INVENTION

The formula contains a combination of nutrients ideal for cardiovascular health. Coenzyme Q10 (CoQ10), tocotrienols and tocopherols (potent forms of vitamin E) promote healthy heart function, provide powerful antioxidant protection and support the immune system in its daily defense of the body. The composition includes CoQ10 with a second component, the second component being d-gamma tocopherol, a tocotrienol or Black Pepper Extract.

The formula may contain a unique tocotrienol complex, derived from the fruit oil of palm trees. Tocotrienols (also known as “super vitamin E”) have been clinically proven to promote healthy cholesterol levels. They inhibit production of cholesterol in the liver and may actually be able to reverse arterial blockage, promoting efficient blood flow supply to the heart muscle.

The formula contains supremely high-quality forms of both CoQ10 and tocotrienols. The CoQ10 is 100% natural, produced using a patented method that results in a CoQ10 identical to the body's own. For tocotrienols, which includes tocotrienols and tocopherols, as well as other beneficial phytonutrients such as plant squalene, phytosterols and carotenoids. A standardized extract of the fruit of Piper nigrum (black pepper), which increases bioavailability of CoQ10 is added.

The formula is enhanced because it contains rice bran oil, which provides additional cholesterol-health benefits and is rich in antioxidants (with one to two percent gamma-oryzanol and tocotrienols). Contains all-natural carob coloring, which is gentle on the digestive tract and provides additional antioxidant benefits

### DETAILED DESCRIPTION OF THE INVENTION

The formula contains supremely high-quality forms of both CoQ10 and tocotrienols. The tocotrienols may be supplied in the form of Vitamin E. The preferred formulation is as follows:

| | | | Specific | |
|---|---|---|---|---|
| Component | Range | | Embodiment | |
| Vitamin E (from high gamma mixed | 1–10,000 | IU | 10 | IU |
| tocopherols and tocotrienol complex) | | | | |
| Coenzyme Q10 | 10–10000 | mg | 100 | mg |
| (ubiquinone USP)(naturally | | | | |
| fermented) | | | | |
| Total Tocopherols & Tocotrienols | 4.5–4600 | mg | 45.8 | mg |
| Typical distribution | | | | |
| d-gamma tocopherol | 2.5–2500 | mg | 25 | mg |
| d-delta tocopherol | 1–1000 | mg | 10 | mg |
| d-alpha tocopherol | 0.68–6800 | mg | 6.8 | mg |
| d-beta tocopherol | 83.4–83400 | mcg | 834 | mcg |
| d-gamma tocotrienol | .17–1700 | mg | 1.7 | mg |
| d-alpha tocotrienol | 92–92000 | mcg | 920 | mcg |
| d-delta tocotrienol | 44–44000 | mcg | 440 | mcg |
| d-beta tocotrienol | 12–12000 | mcg | 120 | mcg |
| Black Pepper Extract | .5–500 | mg | 5 | mg |
| (Piper nigrum)(fruit) | | | | |
| Plant squalene | 80–80,000 | mcg | 800 | mcg |
| Phytosterols complex | 28–28000 | mcg | 280 | mcg |
| Mixed carotenoid complex | 4–400 | mcg | 4 | mcg |

| | Specific | | | | | |
|---|---|---|---|---|---|---|
| Component | Embodiments | | | | | |
| Vitamin E (from high | 10 | IU | 10 | IU | 10 | IU |
| gamma mixed tocopherols | | | | | | |
| and tocotrienol complex) | | | | | | |
| Coenzyme Q10 | 200 | mg | 400 | mg | 60 | mg |
| (ubiquinone USP)(naturally | | | | | | |
| fermented) | | | | | | |
| Total Tocopherols & | 43.2 | mg | 40.6 | mg | 46.9 | mg |
| Tocotrienols | | | | | | |
| Typical distribution | | | | | | |
| d-gamma tocopherol | 21 | mg | 16.9 | mg | 26.6 | mg |
| d-delta tocopherol | 8.4 | mg | 6.8 | mg | 10.6 | mg |
| d-alpha tocopherol | 6.8 | mg | 6.8 | mg | 6.8 | mg |
| d-beta tocopherol | 699 | mcg | 564 | mcg | 887 | mcg |
| d-gamma tocotrienol | 3.4 | mg | 5 | mg | 1 | mg |
| d-alpha tocotrienol | 1.8 | mg | 2.8 | mg | 552 | mcg |
| d-delta tocotrienol | 880 | mcg | 1.3 | mg | 264 | mcg |
| d-beta tocotrienol | 320 | mcg | 480 | mcg | 96 | mcg |
| Black Pepper Extract | 5 | mg | 5 | mg | 5 | mg |
| (Piper nigrum)(fruit) | | | | | | |
| Plant squalene | 1.6 | mg | 2.4 | mg | 480 | mcg |
| Phytosterols complex | 560 | mcg | 840 | mcg | 168 | mcg |
| Mixed carotenoid complex | 8 | mcg | 12 | mcg | 2.4 | mcg |

While the invention has been described with references to preferred embodiments, variations and modifications would be apparent to one of ordinary skill in the art without departing from the spirit of the invention. The invention encompasses such variations and modifications. For instance. The amount of each component can be varied.

For example, CoQ10 levels may decrease with the use of statins so that the maximum level of Vitamin E can be increased to 2,000 IU. This amount was safely used in an Alzheimer's study reported in the New England Journal of Medicine.

I claim:

1. A formulation comprising
Coenzyme Q10 and a second component selected from the group consisting of g-gamma tocopherol, tocotrienol and Black Pepper Extract.

2. The formulation of claim 1 wherein said second component is g-gamma tocopherol.

3. The formulation of claim 1 wherein said second component is tocotrienol.

4. The formulation of claim 1 wherein said second component is Black Pepper Extract.

5. The formulation of claim 4 further comprising 0.5-500 mg Black Pepper Extract.

6. The formulation of claim 5 further comprising 5 mg Black Pepper Extract.

7. The formulation of claim 1 further comprising rice bran oil.

8. A formulation comprising
Tocopherols & Tocotrienols and
Coenzyme Q10.

9. The formulation of claim 8, wherein said Tocopherols & Tocotrienols comprises
2.5-2500 mg d-gamma tocopherol,
1-1000 mg d-delta tocopherol,
0.68-6800 mg d-alpha tocopherol,
83.4-83400 mcg d-beta tocopherol,
0.17-1700 mg d-gamma tocotrienol,
92-92000 mcg d-alpha tocotrienol,
44-44000 mcg d-delta tocotrienol, and
12-12000 mcg d-beta tocotrienol.

10. The formulation of claim 9 wherein said Tocopherols & Tocotrienols comprises
25 mg d-gamma tocopherol,
10 mg d-delta tocopherol,
6.8 mg d-alpha tocopherol,
834 mcg d-beta tocopherol,
1.7 mg d-gamma tocotrienol,
920 mcg d-alpha tocotrienol,
440 mcg d-delta tocotrienol, and
120 mcg d-beta tocotrienol.
